(19) United States
(12) Patent Application Publication
(10) Pub. No.: US 2021/0085816 A1
(43) Pub. Date: Mar. 25, 2021

(54) HYGIENIC MASK-STORAGE CASE

(71) Applicant: DESIZON CO., LTD., Cheongju-si, Chungcheongbuk-do (KR)

(72) Inventors: Young Jin KIM, Cheongju?si, Chungcheongbuk?do (KR); Yoon Soo KIM, Cheongju?si, Chungcheongbuk?do (KR)

(73) Assignee: DESIZON CO., LTD., Cheongju?si, Chungcheongbuk?do (KR)

(21) Appl. No.: 17/050,844

(22) PCT Filed: May 6, 2020
(86) PCT No.: PCT/KR2020/005948

§ 371 Date: Oct. 27, 2020

(30) Foreign Application Priority Data
May 20, 2019 (KR) 10-2019-0058898

(51) Int. Cl. A61L 2/26; A45C 11/24; A45C 15/00; A61L 2/02; A61L 2/238

(52) U.S. Cl. CPC A61L 2/26; A45C 11/24; A45C 2011/007; A61L 2/022; A61L 2/238; A45C 15/00

(57) ABSTRACT

The present disclosure relates to a sanitary mask storage, and more particularly, to a sanitary mask storage having a disposable mask for personal hygiene (including, for example, fine dust and yellow dust masks) dehumidified, antibacterialized, and deodorized when storing the disposable mask, thereby allowing hygienic storage for the disposable mask to be reused several times.

### TECHNICAL FIELD

Embodiments of the present disclosure relates to a sanitary mask storage, and more particularly, to a sanitary mask storage having a disposable mask for personal hygiene (including, for example, fine dust and yellow dust masks) dehumidified, antibacterialized, and deodorized when storing the disposable mask, thereby allowing hygienic storage for the disposable mask to be reused several times.

### DISCUSSION OF RELATED ART

In general, masks, especially sanitary masks, are objects that cover the nose and mouth to prevent germs or dust from entering the respiratory system.

Such a mask prevents people from inhaling cold air directly, which may weaken resistance in the nasal cavity or mouth, and catching a cold and also prevents fine foreign substances including dust from the outside enter the body through the mouth and nose, and accordingly, it is worn to prevent respiratory diseases or occupational diseases such as silicosis or pneumoconiosis.

When a user wearing the mask inhales and exhales, pathogenic bacteria may grow on an inner surface of the mask with odor due to breath or saliva, and dust or other bacteria adhere to an outer surface to reproduce.

As such, due to the user's repetitive breathing and skin contact and contaminants introduced from the outside and adsorbed on the mask, the mask should be used for a short period of time for a single use and then discarded for personal hygiene, but sanitary masks for yellow dust or fine dust which is significantly harmful to human body are more expensive than regular sanitary masks, so it is common to take them off after wearing them (put them in pockets, in bags, on desks, etc.) and reuse them.

However, bacteria may grow on the mask when the used mask is reused after storing them in a pocket, in a bag, or on a desk, so there is a problem that it is harmful to the human body, especially the respiratory system.

In order to solve this problem, a mask storage case has been recently introduced that allows sanitary masks to be sterilized while storing, an example of which is Korean Utility Model Publication No. 20-0348505 (hereinafter “Document 1”).

As illustrated in the accompanying drawing, FIG. 1, Document 1 is related to a mask disinfecting storage case which includes a housing-type upper body open toward the bottom, a housing-type lower body open toward the top and hinge-coupled to the upper body, and a securing hook formed at another side of coupling, characterized in that a function panel 50 formed of silver component is formed at inner side surfaces of the upper body 10 and the lower body 20, and the function panel 50 is secured as a securing means 60 in respective inner surfaces.

The conventional mask case as described above is simply to store masks in the functional panel 50 formed of silver (Al) to sterilize bacterial foreign substances through sterilization and disinfection function of silver.

However, Document 1 has a problem in that it is not possible to fundamentally solve the cause of bacterial propagation because it is impossible to quickly remove moisture infiltration into the mask generated by user's wearing.

In addition, there is a problem in that the mask stored in the case is not fastened and is folded while moving around, such that dehumidification may not be performed particularly in a folded portion, and sterilization is not sufficiently achieved.

Prior literature related to the sanitary mask storage (case) as described above is as follows.
Document 1: Korean Utility Model Publication No. 20-0348505 (Title: sterilization case to keep mask); filing date: Jan. 17, 2004)
Document 2: Korean Utility Model Publication No. 20-2019-0000622 (Title: packaging case of yellow dust mask); filing date: Jul. 2, 2018)

### DETAILED DESCRIPTION OF THE INVENTION

### Technical Objectives

Aspects of embodiments of the present disclosure may be directed to a sanitary mask storage capable of dehumidification, antibacterialization, and deodorization while storing a disposable mask for personal hygiene (including fine dust and yellow dust masks), thereby allowing hygienic storage and reuse.

### Technical Solution to the Problem

According to an embodiment, a sanitary mask storage 10 includes: an accommodating portion 11 including a first accommodating portion 11A and a second accommodating portion 11B defining an accommodating space for a mask 1; a dehumidifier 12 provided at any one of the first and second accommodating portions 11A and 11B and absorbing and removing moisture in the accommodating portion 11; a deodorizer 13 provided at any one of the first and second accommodating portions 11A and 11B and absorbing and removing pollutants and odor particles in the accommodating portion 11; a sterilizer 14 provided at any one of the first and second accommodating portions 11A and 11B and sterilizing the accommodating portion 11 by reaction with bacteria in the accommodating portion 11; and a vent 15 provided at any one of the first and second accommodating portions 11A and 11B and discharging expanding air or gas of the accommodating portion 11.

In some embodiments, the sanitary mask storage 10 may be formed as a housing-type hard case.

In some embodiments, the sanitary mask storage 10 may be formed as a film-type pouch.

In some embodiments, the first accommodating portion 11A may be a case body configured to accommodate the mask 1, and the second accommodating portion 11B may be a cover covering and blocking the case body.

In some embodiments, the first and second accommodating portions 11A and 11B may be two-ply opposing film bodies to be sealed.

In some embodiments, the dehumidifier 12 may include a dehumidifying agent storage 12b allowing a dehumidifying agent 12a to be inserted/withdrawn.

In some embodiments, the deodorizer 13 may include a deodorizing agent storage 13b allowing a deodorizing agent 13a to be inserted/withdrawn.

In some embodiments, the sterilizer 14 may include a sterilizing agent storage 14b allowing the sterilizing agent 14a to be inserted/withdrawn.

In some embodiments, the vent 15 may be a one-way valve body for discharging air and gas in the accommodating portion 11 to the outside.

In some embodiments, a mask securing portion 16 protruding from a portion of one of the first and second accommodating portions 11A and 11B may be further provided, the mask securing portion pressing and securing the accommodated mask 1 to another portion corresponding to the one portion.

In some embodiments, a space of the first accommodating portion 11A may expand, a center portion of the space of the first accommodating portion may be partitioned into a used mask accommodating portion 11′ and a storage mask accommodating portion 11″, and a blocking plate 17 for opening and closing the partitioned portion may be further provided.

### Effects of the Invention

According to various embodiments of the present disclosure, a sanitary mask storage allows dehumidification, antibacterialization, and deodorization of a disposable sanitary mask for personal hygiene (including fine dust and yellow dust masks) while storing it in a hard case or a film pouch, and thus the disposable sanitary mask may be hygienically stored and reused several times.

In addition, embodiments of the present disclosure provide, at one of first and second accommodating portions 11A and 11B, a dehumidifier 12 including silica gel which is a dehumidifying agent for absorbing and removing moisture in an accommodating portion 11 to dehumidify moisture in a mask 1 itself accommodated in the accommodating portion 11 or moisture generated in the accommodating portion 11, thereby preventing propagation of bacteria by moisture and allowing use of mask 1 in a refreshing state.

In addition, embodiments of the present disclosure provide, at one of the first and second accommodating portions 11A and 11B, a deodorizer 13 including bentonite, zeolite, and activated carbon which are deodorizing agents for absorbing and removing odor in the accommodating portion 11 to absorb and remove odor and contaminant on the mask 1 accommodated in an accommodating portion 11 or odor and contaminant generated in the accommodating portion 11, thereby allowing use of mask 1 in a refreshing state.

In addition, embodiments of the present disclosure provide, at one of the first and second accommodating portions 11A and 11B, a sterilizer 14 including components of silver, copper, and tin which are sterilizing agents for reacting with bacteria in the accommodating portion 11 to sterilize bacteria that propagate on the mask 1 itself accommodated in the accommodating portion 11 or in the accommodating portion 11, thereby allowing use of mask 1 in a refreshing and hygienic state.

In addition, embodiments of the present disclosure expand a space in the first accommodating portion 11A, partition a center portion thereof into a used mask accommodating portion 11′ and a storage mask accommodating portion 11″, and further include a blocking plate 17 for opening and closing the partitioned portion, and accordingly, in case of emergency, a used mask may be discarded and replaced with a storage mask.

### BRIEF DESCRIPTION OF DRAWINGS

FIG. 1 is a perspective view illustrating configuration of a conventional mask storage.

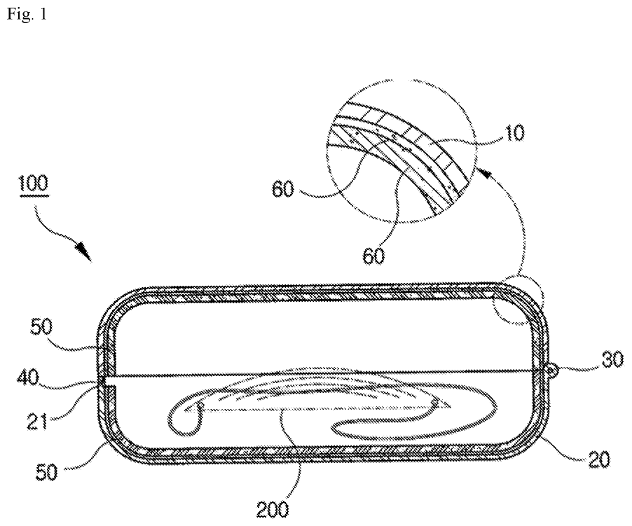

FIG. 2 is a perspective view illustrating an appearance of an embodiment of the present disclosure.

FIG. 3 is an open perspective view illustrating an overall external and internal structure of an embodiment of the present disclosure.

FIG. 4 is a front cross-sectional view illustrating a configuration, a structure and a use state of an embodiment of the present disclosure.

FIG. 5 is an open perspective view illustrating an overall external and internal structure of another embodiment of the present disclosure.

FIG. 6 is a front cross-sectional view illustrating a use state of another embodiment of the present disclosure.

### MODES FOR CARRYING OUT THE INVENTION

The present disclosure will be described with reference to the accompanying drawings presented as described above.

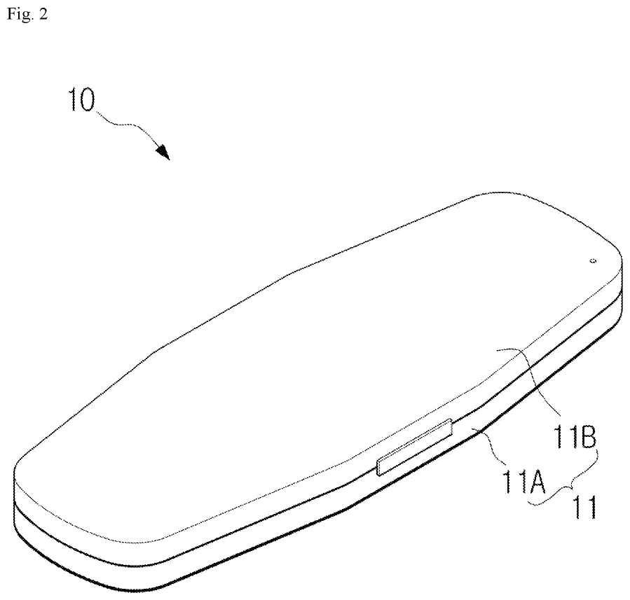

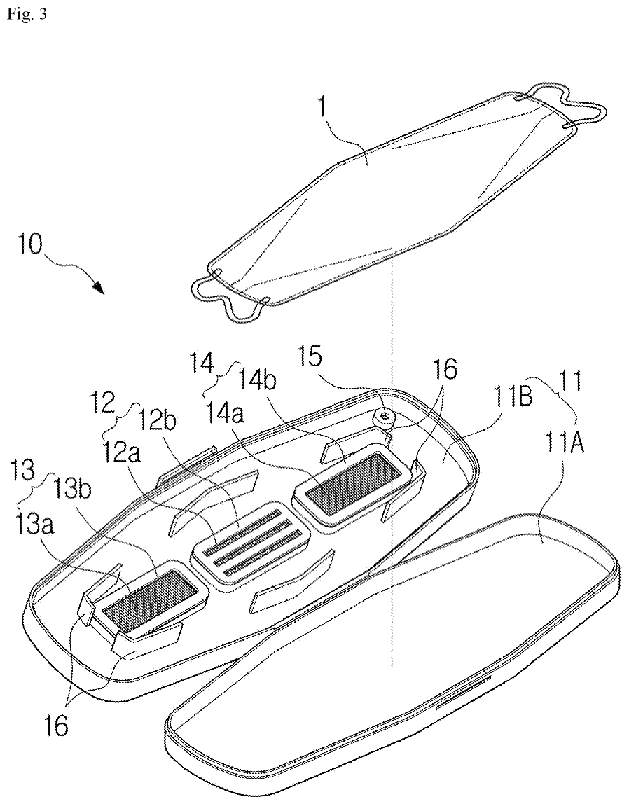

As illustrated FIGS. 2 and 3, a sanitary mask storage 10 according to an embodiment includes, an accommodating portion 11 including a first accommodating portion 11A and a second accommodating portion 11B defining an accommodating space for a mask 1, a dehumidifier 12 provided at any one of the first and second accommodating portions 11A and 11B to absorb and remove moisture in the accommodating portion 11, a deodorizer 13 provided at any one of the first and second accommodating portions 11A and 11B to absorb and remove pollutants and odor particles in the accommodating portion 11, a sterilizer 14 provided at any one of the first and second accommodating portions 11A and 11B to sterilize the accommodating portion 11 by reaction with bacteria in the accommodating portion 11, and a vent 15 provided at any one of the first and second accommodating portions 11A and 11B to discharge expanding air or gas of the accommodating portion 11.

In such a case, the sanitary mask storage 10 according to an embodiment may be a housing-type hard case or a film-type pouch.

In an embodiment, the dehumidifier 12 may include a dehumidifying agent storage 12b accommodating a dehumidifying agent 12a to be inserted/withdrawn.

In such a case, the dehumidifying agent 12a may be a processed dehumidifying agent itself formed by processing any one of silica gel or bentonite or sericite or loess processed in the form of powder or granule or block or gel, or a dehumidifying agent pack composite-filled with any one or more of the processed dehumidifying agents.

In such a case, the dehumidifying agent storage 12b may be an accommodation body provided on a surface of the second accommodating portion 11B opposing the mask 1 and may have a slit or a through hole or a partial opening on a surface thereof opposing the mask 1.

In such a case, the dehumidifying agent storage 12b may include a cover with an opening portion of a slit or a through hole or a partial opening that is coupled in a detachable manner to the accommodation body (a volume body) which is formed by protruding unitarily from the surface of the second accommodating portion 11B opposing the mask 1.

In such a case, the dehumidifying agent storage 12b may be an accommodation body (a film body or a mesh body formed with perforation) unitarily provided on the surface of the second accommodating portion 11B opposing the mask 1 through heat fusion or attachment.

In an embodiment, the deodorizer 13 may include a deodorizing agent storage 13b for accommodating a deodorizing agent 13a to be inserted/withdrawn.

In such a case, in an embodiment, the deodorizing agent 13a may be a processed deodorizing agent itself formed by processing any one of bentonite or zeolite or activated carbon or sericite or loess processed in the form of powder or granule or block or gel, or a deodorizing agent pack composite-filled with any one or more of the processed deodorizing agents.

In such a case, in an embodiment, the deodorizing agent storage 13b may have the same structure as the dehumidifying agent storage 12b.

In an embodiment, the sterilizer 14 may include a sterilizing agent storage 14b for accommodating a sterilizing agent 14a to be inserted/withdrawn.

In such a case, in an embodiment, the sterilizing agent 14a may be silver or/and copper or/and tin itself or a processed body thereof, or a sterilization pack composite-filled with silver or/and copper or/and tin.

In such a case, in an embodiment, the sterilizing agent storage 14b may have the same structure as the dehumidifying agent storage 12b and the deodorizing agent storage 13b.

In an embodiment, the vent 15 may be a one-way valve body for discharging air and gas in the accommodating portion 11 to the outside.

In such a case, in an embodiment, the vent 15, which is a one-way valve, is provided in a space body formed in the second accommodating portion 11B, and the space body may have a through hole defined inside and outside the space body.

In addition, according to an embodiment, a mask securing portion 16 protruding from a portion of one of the first and second accommodating portions 11A and 11B to press and secure the accommodated mask 1 to another portion corresponding to the one portion is further provided.

In such a case, the mask securing portion 16 may be a pressing protrusion protruding from the second accommodating portion 11B to press an entire or part of an edge of the mask 1.

Hereinafter, an operation of the present disclosure will be described.

An embodiment of the present disclosure provides a sanitary mask storage 10 including an accommodating portion 11 including a first accommodating portion 11A and a second accommodating portion 11B defining an accommodating space for a mask 1, a dehumidifier 12 provided at any one of the first and second accommodating portions 11A and 11B to absorb and remove moisture in the accommodating portion 11, a deodorizer 13 provided at any one of the first and second accommodating portions 11A and 11B to absorb and remove pollutants and odor particles in the accommodating portion 11, a sterilizer 14 provided at any one of the first and second accommodating portions 11A and 11B to sterilize the accommodating portion 11 by reaction with bacteria in the accommodating portion 11, and a vent 15 provided at any one of the first and second accommodating portions 11A and 11B to discharge expanding air or gas of the accommodating portion 11.

Here, the sanitary mask storage 10 according to an embodiment is preferably a housing-type hard case so that the mask 1 may be stably stored from external pressure and is also preferably a film-type pouch to store the mask 1 in a slim size or volume to carry it around easily.

In such a case, in a case where the mask storage 10 is a case made of a housing-type hard material, it is preferable that the first accommodating portion 11A is a case body accommodating the mask 1 and the second accommodating portion 11B is a cover to cover the case body and block, it is preferable that one side of the first accommodating portion 11A, the case body, and one side of the second accommodating portion 11B, the cover, are coupled to each other by a hinge or connected into an integrated part by a connector during injection molding to be open and closed, and it is also preferable that another side of the first accommodating portion 11A, the case body, and another side of the second accommodating portion 11B, the cover, are formed with a coupling protrusion and a depression, respectively, to maintain a locked state when closed.

In such a case, when the mask storage 10 is a film-type pouch, it is preferable that the first accommodating portion 11A and the second accommodating portion 11B are two-ply opposing film bodies whose opposite ends are sealed.

In such a case, a line zipper is provided in a sealed portion on one side, and after using the mask 1, the mask 1 is placed in the accommodating portion 11 and the line zipper is closed for storage.

In an embodiment, the dehumidifier 12 includes the dehumidifying agent storage 12b for accommodating the dehumidifying agent 12a to be inserted/withdrawn, and in an embodiment, the dehumidifying agent 12a may be a processed dehumidifying agent itself formed by processing any one of silica gel or bentonite or sericite or loess processed in the form of powder or granule or block or gel, or a dehumidifying agent pack composite-filled with any one or more of the processed dehumidifying agents, and in an embodiment, the dehumidifying agent storage 12b is an accommodation body provided on the surface of the second accommodating portion 11B opposing the mask 1 and may have a slit or a through hole or a partial opening on a surface of the dehumidifying agent storage 12b opposing the mask 1.

The silica gel is a high-porosity moisture absorbent including silicon dioxide (SiO₂) as its main component and has excellent effect of adsorbing moisture or small molecules, thus adsorbing water molecules or other substances in the air, to confine them in the space between particles and absorb moisture.

In addition, when the bentonite contacts moisture, electrons and molecular components rapidly change to generate electric charges, and in such a case, the generated charges absorb and trap toxins, impurities, heavy metals and other internal contaminants.

In such a case, it is preferable that the dehumidifying agent storage 12b includes a cover with an opening portion of a slit or a through hole or a partial opening that is coupled in a detachable manner to the accommodation body (a volume body) which is formed by protruding unitarily from the surface of the second accommodating portion 11B opposing the mask 1.

In such a way, the dehumidifying agent 12a accommodated in the accommodation body may be inserted/withdrawn, and the dehumidifying agent 12a may be replaced.

In addition, in such a case, it is also preferable that the dehumidifying agent storage 12b is an accommodation body (a film body or a mesh body formed with perforation) unitarily provided on the surface of the second accommodating portion 11B opposing the mask 1 through heat fusion or attachment, and although not illustrated, it is useful when the accommodating portion 11 is made of a film-type pouch.

In an embodiment, the deodorizer 13 includes the deodorizing agent storage 13b for accommodating the deodorizing agent 13a to be inserted/withdrawn, and in an embodiment, the deodorizing agent may be a processed deodorizing agent itself formed by processing any one of bentonite or zeolite or activated carbon or sericite or loess processed in the form of powder or granule or block or gel, or a deodorizing agent pack composite-filled with any one or more of the processed deodorizing agents, and in an embodiment, the deodorizing agent storage 13b is an accommodation body provided on the surface of the second accommodating portion 11B opposing the mask 1 and may have a slit or a through hole or a partial opening on a surface of the deodorizing agent storage 13b opposing the mask 1.

The bentonite has a function of moisture absorption, but there are a large amount of natural minerals with positive ions on the surface, which have exchangeability with cations such as odor particles and pollutants on the mask 1 itself and in the accommodating portion 11, thus attracting and adsorbing the odor particles and pollutants and discharge cations beneficial to the human body toward the mask 1 and the accommodating portion 11.

In addition, since zeolite includes exchangeable cations in its crystal structure which are easily exchanged with other cations and also serves functions as in bentonite, zeolite may remove harmful substances on the mask 1 and in the accommodating portion 11, concentrates and recovers effective components, and serves antibacterial function by replacing part of cations with anion.

In addition, the activated carbon removes organic substances from the water by attraction and adsorption power of an active surface. The most important factors for the removal or adsorption of organic substances from the water may be physical properties such as high specific surface area and pore structure and chemical properties of the active surface of the activated carbon.

In such a case, it is preferable that the deodorizing agent storage 13b includes a cover with an opening portion of a slit or a through hole or a partial opening that is coupled in a detachable manner to the accommodation body (a volume body) which protrudes unitarily from the surface of the second accommodating portion 11B opposing the mask 1.

In such a way, the deodorizing agent 13a accommodated in the accommodation body may be inserted/withdrawn, and the deodorizing agent 13a may be replaced.

In addition, in such a case, it is also preferable that the deodorizing agent storage 13b is an accommodation body (a film body or a mesh body formed with perforation) unitarily provided on the surface of the second accommodating portion 11B opposing the mask 1 through heat fusion or attachment, and although not illustrated, it is useful when the accommodating portion 11 is made of a film-type pouch.

In an embodiment, the sterilizer 14 includes the sterilizing agent storage 14b for accommodating the sterilizing agent 14a to be inserted/withdrawn, and in an embodiment, the sterilizing agent 14a may be silver (including silver nanostructure) or/and copper or/and tin itself or a processed body thereof, or a sterilization pack filled with silver or/and copper or/and tin, and in an embodiment, the sterilizing agent storage 14b is an accommodation body provided on a surface of the second accommodating portion 11B opposing the mask 1 and may have a slit or a through hole or a partial opening on a surface of the sterilizing agent storage 14b opposing the mask 1.

Silver (Al) easily becomes silver ions (Ag⁺) when it encounters moisture, and when silver ions meet bacteria, it inhibits enzyme that controls the respiration of bacteria, such that the bacteria cannot breathe and die. Silver (Ag) bonds with oxygen molecules (O₂) and releases active oxygen, which serves a strong oxidizing action, so it has a strong sterilizing power in proportion to the oxidation action.

In such a case, it is preferable that the sterilizing agent storage 14b includes a cover with an opening portion of a slit or a through hole or a partial opening that is coupled in a detachable manner to the accommodation body (a volume body) which is formed by protruding unitarily from the surface of the second accommodating portion 11B opposing the mask 1.

In such a way, the sterilizing agent 14a accommodated in the accommodation body may be inserted/withdrawn, and the sterilizing agent 14a may be replaced. In addition, in such a case, it is also preferable that the sterilizing agent storage 14b is an accommodation body (a film body or a mesh body formed with perforation) unitarily provided on the surface of the second accommodating portion 11B opposing the mask 1 through heat fusion or attachment, and although not illustrated, it is useful when the accommodating portion 11 is made of a film-type pouch.

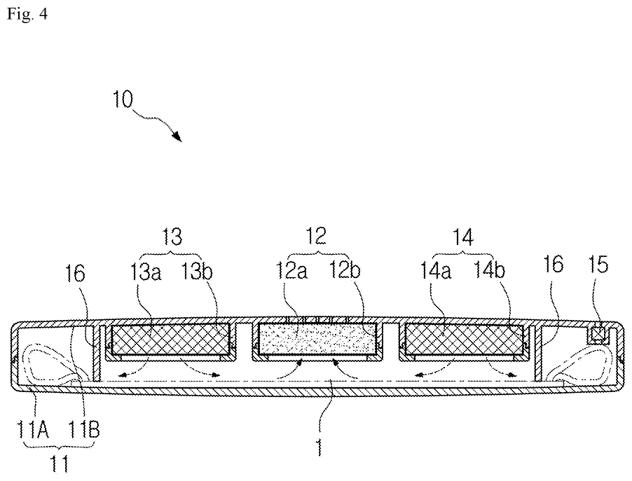

As such, as illustrated in FIG. 4, a sanitary mask storage according to an embodiment allows dehumidification, antibacterialization, and deodorization of a disposable sanitary mask for personal hygiene (including fine dust and yellow dust masks) while storing it in a hard case or a film pouch, and thus the disposable sanitary mask may be hygienically stored and reused several times.

In addition, according to an embodiment, the dehumidifier 12 including silica gel which is a dehumidifying agent for absorbing and removing moisture in the accommodating portion 11 is provided at one of first and second accommodating portions 11A and 11B to dehumidify moisture on the mask 1 itself accommodated in the accommodating portion 11 or moisture generated in the accommodating portion 11, thereby preventing propagation of bacteria by moisture and thus allowing use of mask 1 in a refreshing state.

In addition, according to an embodiment, the deodorizer 13 including bentonite, zeolite, and activated carbon which are a deodorizing agent for absorbing and removing odor in the accommodating portion 11 is provided at one of the first and second accommodating portions 11A and 11B to absorb and remove odor and contaminant of the mask 1 accommodated in the accommodating portion 11 or odor and contaminant generated in the accommodating portion 11, thereby allowing use of mask 1 in a refreshing state.

In addition, according to an embodiment, the sterilizer 14 including components of silver, copper, and tin which are sterilizing agents for reacting with bacteria in the accommodating portion 11 is provided at one of first and second accommodating portions 11A and 11B to sterilize bacteria that propagate on the mask 1 itself accommodated in the accommodating portion 11 or in the accommodating portion 11, thereby allowing use of mask 1 in a refreshing and hygienic state.

In addition, according to an embodiment, the vent 15 is a one-way valve body for discharging air and gas in the accommodating portion 11 to the outside, and in an embodiment, the vent 15 is provided in a space body formed in the second accommodating portion 11B, and the space body may have a through hole formed inside and outside the space body.

It is preferable that the dehumidifier 12, the deodorizer 13, and the sterilizer 14 according to embodiments as described above are provided in a row at intervals at the second accommodating portion 11B, but it is also preferable that the sterilizer 14 is provided as a coating layer attachment or a stack assembly provided over an entire inner surface of the accommodating portion 11 formed by the first and second accommodating portions 11A and 11B.

As described above, as the sterilizer 14 is provided over the entire inner surface of the accommodating portion 11 formed by the first and second accommodating portions 11A and 11B, the sterilizing force is uniformly applied to the entire mask 1.

According to an embodiment, it is preferable that the mask securing portion 16 protruding from a portion of one of the first and second accommodating portions 11A and 11B to press and secure the accommodated mask 1 to another portion corresponding to the one portion is further provided, and in such a case, it is preferable that the mask securing portion 16 is a pressing protrusion protruding from the second accommodating portion 11B to press an entire or part of an edge of the mask 1.

In such a way, the mask securing portion 16 presses and holds the mask 1 which is spread and stored in the accommodating portion 11 to prevent moving of the mask 1. In addition, by allowing the surface of the mask 1 to be spread evenly, dehumidification, deodorization, and sterilization may be uniformly performed.

In addition, in a state that the mask securing portion 16 presses along the edge of the mask 1 as described above, the dehumidifier 12, the deodorizer 13, and the sterilizer 14 located inwardly of the mask securing portion 16 are allowed to intensively act on the mask 1.

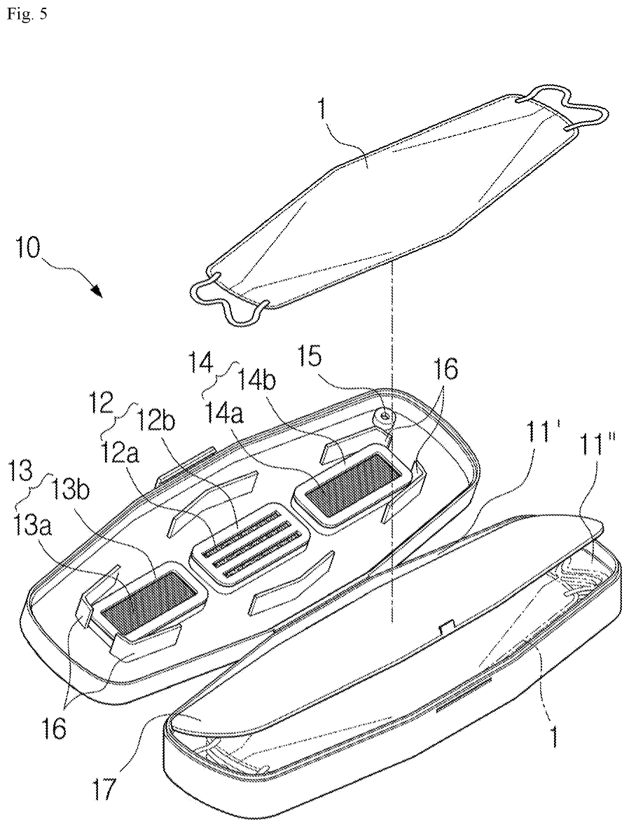

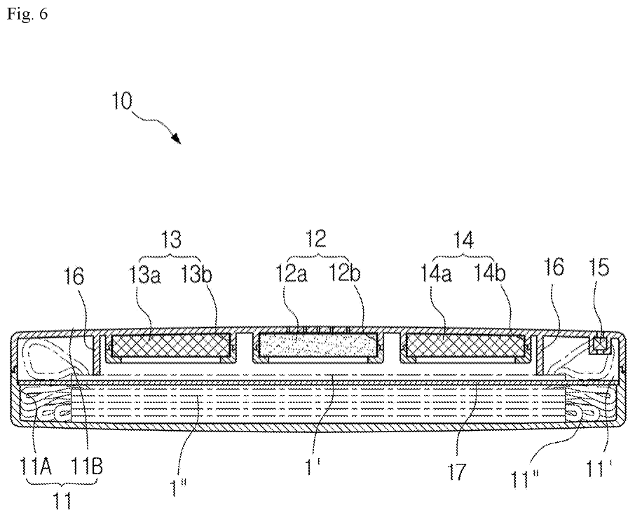

In an embodiment, as illustrated in FIGS. 5 and 6, it is also preferable that a space in the first accommodating portion 11A expands, a center portion of the space in the first accommodating portion 11A is partitioned into the used mask accommodating portion 11′ and the storage mask accommodating portion 11″, and the blocking plate 17 for opening and closing the partitioned portion is further provided.

By expanding the space in the first accommodating portion 11A, partitioning the center portion thereof into the used mask accommodating portion 11′ and the storage mask accommodating portion 11″, and further providing the blocking plate 17 for opening and closing the partitioned portion in such a way, it is possible to store the used mask 1′ in the used mask accommodating portion 11′ and store a plurality of unused masks 1′ in the storage mask accommodating portion 11″, and accordingly, in case of emergency, the used mask may be discarded and replaced with the storage mask.

As described above, the present disclosure has been described and illustrated in connection with a preferred embodiment for illustrating the principle of the present disclosure, but the present disclosure is not limited to the configuration and operation as illustrated and described as such.

In addition, it will be well understood by those skilled in the art that many changes and modifications may be made to the present disclosure without departing from the spirit and scope of the appended claims. Accordingly, all such appropriate changes and modifications and equivalents should be considered to be within the scope of the present disclosure.

1. A sanitary mask storage, comprising:
an accommodating portion comprising a first accommodating portion and a second accommodating portion defining an accommodating space for a mask;
a dehumidifier provided at any one of the first and second accommodating portions and absorbing and removing moisture in the accommodating portion;
a deodorizer provided at any one of the first and second accommodating portions and absorbing and removing pollutants and odor particles in the accommodating portion;
a sterilizer provided at any one of the first and second accommodating portions and sterilizing the accommodating portion by reaction with bacteria in the accommodating portion; and
a vent provided at any one of the first and second accommodating portions and discharging expanding air or gas of the accommodating portion.

2. The sanitary mask storage of claim 1, formed as a housing-type hard case or a film-type pouch.

3. The sanitary mask storage of claim 1, wherein the first accommodating portion is a case body configured to accommodate the mask, and
the second accommodating portion is a cover covering and blocking the case body.

4. The sanitary mask storage of claim 1, wherein the first and second accommodating portions are two-ply opposing film bodies to be sealed.

5. The sanitary mask storage of claim 1, wherein the dehumidifier comprises a dehumidifying agent storage allowing a dehumidifying agent to be inserted/withdrawn.

6. The sanitary mask storage of claim 5, wherein the dehumidifying agent is:
a processed dehumidifying agent itself formed by processing any one of silica gel or bentonite or sericite or loess processed in the form of powder or granule or block or gel, or
a dehumidifying agent pack composite-filled with any one or more of the processed dehumidifying agents.

7. The sanitary mask storage of claim 5, wherein the dehumidifying agent storage is an accommodation body provided on a surface of the second accommodating portion opposing the mask and has a slit or a through hole or a partial opening on a surface of the dehumidifying agent storage opposing the mask.

8. The sanitary mask storage of claim 1, wherein the deodorizer comprises a deodorizing agent storage allowing a deodorizing agent to be inserted/withdrawn.

9. The sanitary mask storage of claim 8, wherein the deodorizing agent is:
a processed deodorizing agent itself formed by processing any one of bentonite or zeolite or activated carbon or sericite or loess processed in the form of powder or granule or block or gel, or
a deodorizing agent pack composite-filled with any one or more of the processed deodorizing agents.

10. The sanitary mask storage of claim 8, wherein the deodorizing agent storage is an accommodation body provided on a surface of the second accommodating portion opposing the mask and has a slit or a through hole or a partial opening on a surface of the deodorizing agent storage opposing the mask.

11. The sanitary mask storage of claim 1, wherein the sterilizer comprises a sterilizing agent storage allowing the sterilizing agent to be inserted/withdrawn.

12. The sanitary mask storage of claim 11, wherein the sterilizing agent is silver or copper or tin itself or a processed body thereof, or
a sterilization pack filled with silver or copper or tin.

13. The sanitary mask storage of claim 11, wherein the sterilizing agent storage is an accommodation body provided on a surface of the second accommodating portion opposing the mask and has a slit or a through hole or a partial opening on a surface of the sterilizing agent storage opposing the mask.

14. The sanitary mask storage of claim 1, wherein the dehumidifier, the deodorizer, and the sterilizer are provided in a row at intervals at the second accommodating portion.

15. The sanitary mask storage of claim 1, wherein the sterilizer is provided as a coating layer or a stack assembly provided over an entire inner surface of the accommodating portion formed by the first and second accommodating portions.

16. The sanitary mask storage of claim 1, further comprising a mask securing portion protruding from a portion of one of the first and second accommodating portions, the mask securing portion pressing and securing the accommodated mask to another portion corresponding to the one portion.

17. The sanitary mask storage of claim 1, wherein a space of the first accommodating portion expands, a center portion of the space of the first accommodating portion is partitioned into a used mask accommodating portion and a storage mask accommodating portion, and
the sanitary mask storage further comprises a blocking plate for opening and closing the partitioned portion.
